(19) United States
(12) Patent Application Publication
(10) Pub. No.: US 2022/0259257 A1
(43) Pub. Date: Aug. 18, 2022

(54) PROCESS FOR THE PREPARATION OF FULVESTRANT

(71) Applicant: FARMABIOS S.P.A., Gropello Cairoli (PV) (IT)

(72) Inventors: ROBERTO AROSIO, Civate (LC) (IT); MAURO GABOARDI, Novara (NO) (IT); FRANCESCO ZERILLI, Pavia (PV) (IT)

(73) Assignee: FARMABIOS S.P.A., Gropello Cairoli (PV) (IT)

(21) Appl. No.: 17/592,985

(22) Filed: Feb. 4, 2022

(30) Foreign Application Priority Data
Feb. 12, 2021 (IT) 102021000003176

(51) Int. Cl. C07J 31/00

(52) U.S. Cl. CPC C07J 31/006

(57) ABSTRACT

The present invention relates to a process for the preparation of Fulvestrant which includes recovering the by-products deriving from the oxidation of the corresponding sulfide and then subjecting the by-products to a reduction reaction in the presence of specific reducing agents.

### CROSS-REFERENCE TO RELATED APPLICATIONS

This application claims the benefit of priority from Italian Patent Application

No. 102021000003176 filed on Feb. 12, 2021, the contents of which are incorporated herein by reference.

### FIELD OF THE INVENTION

The present invention relates to a process for the preparation of Fulvestrant, comprising the recovery of the by-products deriving from the oxidation of the corresponding sulfide, which are subjected to a reduction reaction in the presence of specific reducing agents.

### BACKGROUND OF THE INVENTION

Fulvestrant is an antiestrogen used in postmenopausal women with locally advanced or metastatic estrogen receptor positive breast cancer. The active ingredient Fulvestrant, also known as (7α,17β)-7-[9-[(4,4,5,5,5-pentafluoropentyl) sulfinyl]nonyl]estra-1,3,5(10)-triene-3,17-diol, has the following formula (I)

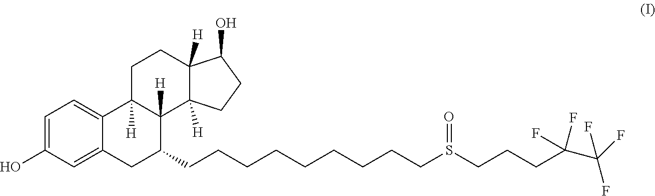

and is marketed as a medicinal product in the form of a solution for intramuscular injection under the brand name Faslodex®.

Fulvestrant in its pharmaceutical form is a mixture of two epimeric diastereomers at the sulfur atom. These two diastereomers are identified as Fulvestrant sulfoxide A and Fulvestrant sulfoxide B.

For use in therapy, the regulatory authorities require to control the amount of each diastereomer in the finished product, maintaining a ratio between isomer A and isomer B between 42-48/58-52.

Several processes are known for the preparation of Fulvestrant. For example, U.S. Pat. No. 4,659,516 discloses a process for the preparation of Fulvestrant according to Scheme 1 reported below:

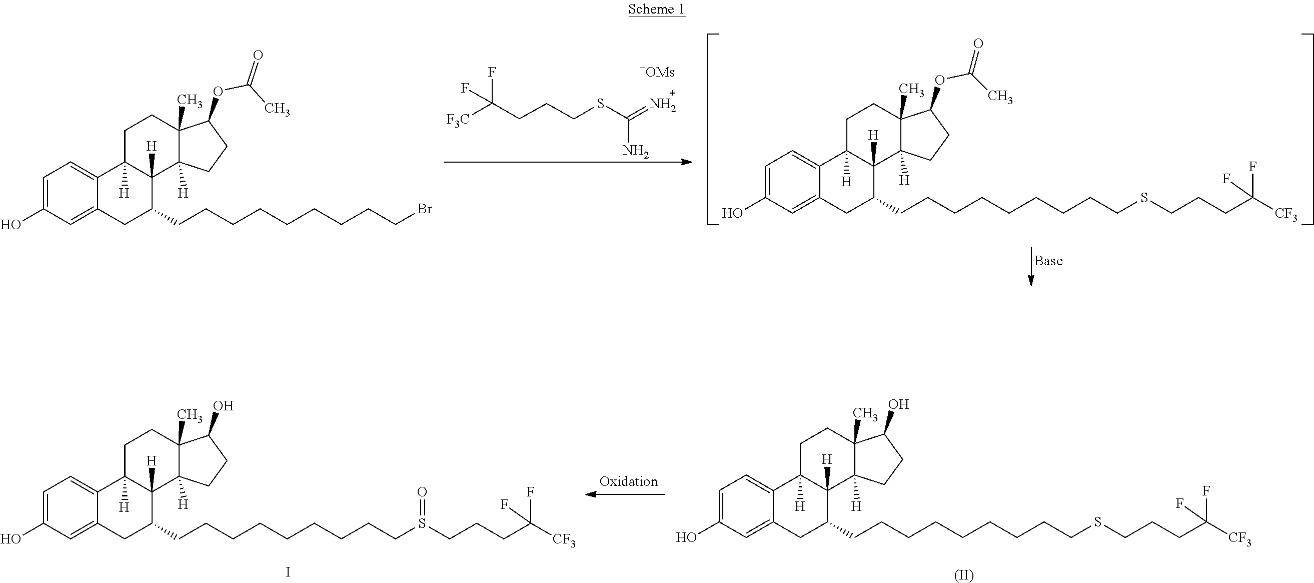

The crude Fulvestrant (I) obtained by oxidation of the corresponding sulfide (II), is purified by known techniques such as chromatography on silica and crystallization from ethyl acetate to isolate Fulvestrant having an isomeric ratio A/B between 42-48/58-52.

However, in addition to obtaining the desired Fulvestrant, the above process presents the problem of the formation of significant quantities of some by-products deriving from the oxidation reaction, such as the corresponding sulfone and Fulvestrant having an isomeric ratio A/B outside the permitted limit that comes from the chemical synthesis obtained in a 50/50 ratio with respect to the Fulvestrant of interest. This implies that after purification, the product fraction falls within the diastereosomeric ratio specifications reported above, while the recovery fractions, isolated after the purification of the crude Fulvestrant, contain large quantities of the aforementioned diastereoisomers in a ratio of about 60/40. These recovery fractions are, therefore, not suitable for commercial purposes, and are normally discarded, thus lowering the overall yield of the process.

CN 106146599 proposes a solution to the problem, i.e. a process for the preparation of Fulvestrant comprising the recycling of the recovery fractions which are subjected to a reduction reaction in the presence of strong reducing agents to obtain the corresponding sulfide and subsequent oxidation to form the desired Fulvestrant. In particular, in this document, the reduction reaction is carried out by various methodologies, such as catalytic hydrogenation, acid-catalyzed metal reaction, borohydride in the presence of a Lewis acid, lithium aluminum hydride or diisobutyl aluminum hydride, sodium borohydride-iodine or boranes, or a combination of dichlorosulfoxide-triphenyl phosphine or oxalyl chloride-triphenyl phosphine.

Similarly, the patent CN 107698647 describes a preparation of Fulvestrant comprising a reduction reaction of the recycled recovery fractions with reducing agents such as sodium bromide or sodium iodide in the presence of an acid such as HBr, HI, BF₃.Et₂O or pTSA. The sulfide thus obtained is then oxidized to lead to the desired Fulvestrant.

However, the prior art makes use of very strong reducing reagents or strongly acidic conditions which could lead to the degradation of the Fulvestrant molecule and therefore to the formation of numerous impurities.

Therefore, there is a need for an effective and safe process of preparation of Fulvestrant that does not use strong reducing reagents.

### SUMMARY OF THE INVENTION

The inventors have now discovered a process for the preparation of Fulvestrant that does not have the drawbacks of the known methods. The process includes recycling the oxidation by-products, which leads to the recovery of about 50-70% of the Fulvestrant from the recovery fraction. In the description of the invention below, a reaction produces crude Fulvestrant, which is purified to Fulvestrant sulfoxide A and Fulvestrant sulfoxide B in the desired ratio of 42-48/58-52. Fulvestrant having the desired ratio is referred to below as product Fulvestrant or simply Fulvestrant.

The chromatographic purification of crude Fulvestrant also produces a fraction of Fulvestrant outside the desired ratio of 42-48/58-52 as well as a sulfone fraction. Fulvestrant outside the desired ratio is referred to below as waste Fulvestrant. The fraction of the reaction product that contains waste Fulvestrant and sulfone is referred to below as the reaction by-product.

An object of the present invention is therefore an improvement of the process for the preparation of a mixture of diastereomer A and diastereomer B in a desired ratio of 42-48/58-52.

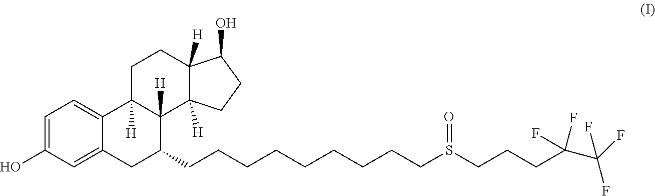

Product Fulvestrant can be prepared by oxidizing sulfide (II) by known methods, such as the oxidation reactions described in U.S. Pat. No. 4,659,516:

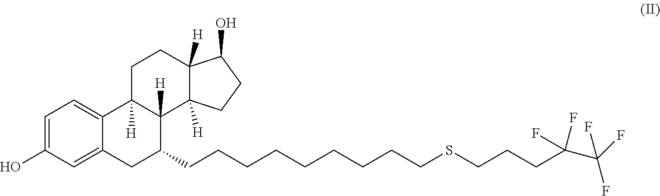

These reactions also produce waste Fulvestrant having a ratio of diastereomer A and diastereomer B outside the range of 42-48/58-52.

It is also known to recycle the waste Fulvestrant by, for example, a reduction reaction in the presence of strong reducing agents to obtain the corresponding sulfide having formula (II) and subsequent oxidation to form additional product Fulvestrant.

In the improvement of the present invention, the reduction reaction takes place in the presence of a reducing agent selected from one or more of sodium bisulfate, sodium metabisulfite, and diiron-nonacarbonyl phenylsilane. The process of the present invention is represented in Scheme 2 below.

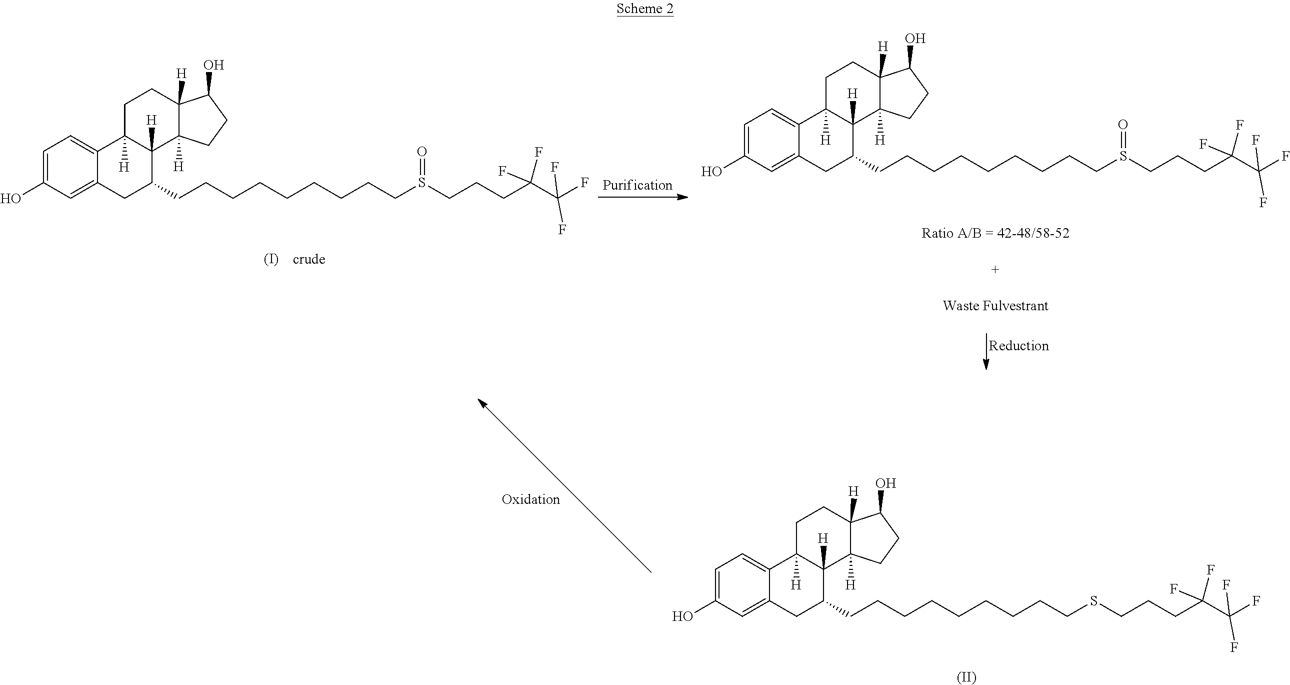

The purification of the crude Fulvestrant is carried out using techniques well known to those skilled in the art, such as silica chromatography and subsequent crystallization in a suitable solvent.

In particular, the first purification step is the chromatographic separation of crude Fulvestrant from the sulfone impurity present in significant quantities in the head fractions of the column.

The subsequent fractions containing the desired product Fulvestrant are subjected to crystallization, preferably in ethyl acetate, to eliminate the waste Fulvestrant having an unsuitable A/B isomeric ratio, i.e. about 60/40. These two purification steps lead to obtaining about 70% overall yield of product Fulvestrant.

Therefore, in order to recover greater quantities of product, the reaction by-products constituted by the head fractions of the column enriched in sulfone and the crystallization mother liquors containing waste Fulvestrant are combined and recycled,

### DETAILED DESCRIPTION OF THE INVENTION

According to the present invention, waste Fulvestrant is subjected to a reduction reaction so as to restore the sulfide (II).

Typically, reaction by-product contains about 90% of waste Fulvestrant as well as about 10% of sulfone (III) of formula:

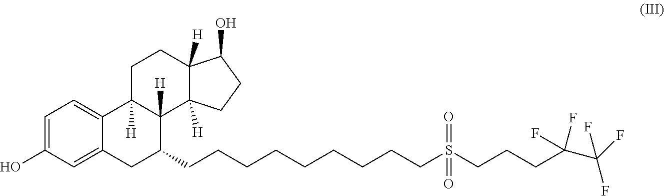

Preferably, the waste Fulvestrant is dissolved in an organic solvent such as for example toluene, tetrahydrofuran, chloroform, acetonitrile, methyl tetrahydrofuran, dioxane. Dissolution is conveniently carried out at room temperature. A reducing agent selected from sodium bisulfite, sodium metabisulfite, and diiron-nonacarbonyl phenylsilane is added to this solution. According to a preferred embodiment, when a reducing agent selected from sodium bisulfite and sodium metabisulfite is used, a catalytic amount of iodine is added to the reaction mixture.

The reducing agent is preferably introduced into the reaction mixture in a molar quantity between 1.0 and 2.0 equivalents with respect to the molar quantity of waste Fulvestrant, more preferably between 1.0 and 1.5 equivalents. Furthermore, according to the present invention, this reducing agent can be added to the reaction mixture as a solid or as an aqueous solution.

Preferably, the reducing agent is used as an aqueous solution as it is commercially available. According to the invention, said aqueous solution has a concentration between 10 and 45%, preferably 40%. After the addition of the reducing agent, the reaction mixture is heated to reflux of the solvent and stirred, generally between 2 and 18 hours, until total conversion of the sulfoxide to sulfide (II) is obtained.

The reducing agents identified by the Applicant are mild agents and particularly suitable for the reduction of waste Fulvestrant for the preparation of sulfide (II). These agents in fact allow the selective conversion of the waste Fulvestrant into the sulfide (II), without degrading or reducing the sulfone (III) present along with the waste Fulvestrant in the reaction by-product.

Furthermore, unlike other mild reducing agents tested by the Applicant, such as sodium sulfite, for example, the reducing agents used in the present invention bring the reduction reaction to completion. A reaction mixture is then obtained which can be subsequently purified to isolate the sulfide (II).

In fact, as it is well known to the skilled person, the mixture can be subjected to various purification operations in order to separate the sulfide (II) from the sulfone (III), such as silica chromatography. Preferably, said mixture is purified by means of a chromatographic column to isolate the sulfide (II) free from the sulfone (III).

According to a preferred embodiment the sulfide (II) obtained with the process of the present invention is subsequently subjected to an oxidation reaction to prepare Fulvestrant (I). The experimental conditions for carrying out this reaction are those known to the skilled person. See above.

In particular, the residue containing the sulfide (II) is dissolved in at least one organic solvent, such as for example tetrahydrofuran, methanol, and mixtures thereof as described in the cited prior art. Preferably, said residue is dissolved in a mixture of tetrahydrofuran and methanol. The solution thus obtained is cooled to a temperature of about 5° C. and an oxidizing agent is added.

Examples of oxidizing agents according to the present invention are sodium (meta) periodate and meta-chloroperbenzoic acid. Generally, after 15-18 hours, the reaction is complete and a residue containing additional crude Fulvestrant is obtained. The additional crude Fulvestrant thus obtained is then purified by techniques known in the art, such as silica chromatography and crystallization from ethyl acetate to provide additional product Fulvestrant.

As is clear to the expert in the field, it is therefore possible to isolate again the recovery fractions resulting from the purification of the raw reaction that still contain waste Fulvestrant not suitable for commercial purposes and, in addition, sulfone (III). The recovery of these fractions can be again subjected to reduction and subsequent oxidation one or more times depending on the quantities to be purified.

Thanks to the process of the present invention and in particular to the use of specific reducing agents, the Applicant has managed to recover 50-70% of the waste Fulvestrant which was previously lost in the final crystallization and in the mixed chromatographic fractions with a significantly higher yield than the process described in U.S. Pat. No. 4,659,516, which does not include the recycling step.

A further object of the present invention is the Fulvestrant obtained by the process of the present invention.

### EXAMPLES

The present invention will now be illustrated by means of some examples, which should not be seen as limiting the scope of the invention.

### Example 1: Procedure for Obtaining Waste Fulvestrant

The fractions of the chromatographic column containing Fulvestrant with the diastereoisomeric ratio not complying with the specifications and enriched with sulfone and the mother liquors of final crystallization are combined. The solvent is distilled under vacuum to a residue. The residue is added with toluene and the mixture is heated up to 70° C. It is cooled to 20° C. and the crystallization of the product is obtained. The product is filtered on Buchner and washed with toluene. The product is dried in an oven at 50° C. under vacuum for 15 h.

### Examples of Reduction from Sulfoxide to Sulfide

### Example 2

Waste Fulvestrant (5 g, 8.24 mmol) is loaded into a 3-necked flask equipped with a mechanical stirrer, thermometer and condenser. Chloroform (25 ml) is charged under nitrogen and there is complete dissolution. Sodium metabisulfite (1.67 g, 8.8 mmol) and iodine (0.20 g, 0.8 mmol) are added to the solution. The reaction mixture is heated under reflux and monitored over time. After 18 h the reaction is complete (conversion>99%). The reaction mixture is cooled to 25±5° C. and demineralized water (15 ml) is added. The phases are separated and the organic phase is washed once with a saturated sodium chloride solution (5 ml). The phases are separated and the organic phase is concentrated under vacuum to a residue. About 4.5 g of product are obtained as thick oil.

### Example 3

Waste Fulvestrant (20 g, 32.97 mmol) is loaded into a 3-necked flask equipped with a mechanical stirrer, thermometer and condenser. THF (100 ml) is charged under nitrogen and there is complete dissolution. Sodium metabisulfite (6.9 g, 36.26 mmol) and iodine (0.83 g, 3.3 mmol) are added to the solution. The reaction mixture is heated under reflux and monitored over time. After 8 h the reaction is complete (conversion>99%). The reaction mixture is cooled to 25±5° C. and demineralized water (60 ml) and toluene (100 ml) are added. The phases are separated and the organic phase is washed once with a saturated sodium chloride solution (5 ml). The phases are separated and the organic phase is dehydrated with magnesium sulphate and concentrated under vacuum to a residue. About 20.3 g of product are obtained as thick oil.

### Example 4

Waste Fulvestrant (10 g, 16.5 mmol) is loaded into a 3-necked flask equipped with a mechanical stirrer, thermometer and condenser. THF (50 ml) is charged under nitrogen and there is complete dissolution. Sodium bisulfite (1.89 g, 18 mmol) and iodine (0.42 g, 1.65 mmol) are added to the solution. The reaction mixture is heated under reflux and monitored over time. After 3 hours and 30 minutes the reaction is complete (conversion>99%). The reaction mixture is cooled to 25±5° C. and demineralized water (60 ml) and toluene (100 ml) are added. The phases are separated and the organic phase is washed once with a saturated sodium chloride solution (5 ml). The phases are separated and the organic phase is dehydrated with magnesium sulphate and concentrated under vacuum to a residue. About 4.8 g of product are obtained as thick oil.

### Example 5

Waste Fulvestrant (5 g, 8.24 mmol) is loaded into a 3-necked flask equipped with a mechanical stirrer, thermometer and condenser. Toluene (25 ml) is charged under nitrogen and there is complete dissolution. Diironnonacarbonyl (0.3 g, 0.82 mmol) and phenyl silane (1 ml, 8.24 mmol) are added to the solution. The reaction mixture is heated to 100° C. and monitored over time. After 3 hours and 30 minutes the reaction is complete. The reaction mixture is cooled to 25±5° C. and the solution is filtered on a panel of tonsil earth. It is washed with toluene and evaporated under vacuum to a residue. About 6 g of product are obtained as thick oil (containing phenylsilane).

### Example 6

Waste Fulvestrant (50 g, 82.4 mmol) is loaded into a 3-necked flask equipped with a mechanical stirrer, thermometer and condenser. THF (250 ml) is charged under nitrogen and there is complete dissolution. 40% sodium bisulfite (30 g, 115 mmol) and iodine (2.08 g, 8.2 mmol) are added to the solution. The reaction mixture is heated under reflux and monitored over time. After 2 h the reaction is complete (conversion>99%). The reaction mixture is cooled to 25±5° C. and demineralized water (60 ml) and toluene (100 ml) are added. The phases are separated and the organic phase is washed once with a saturated sodium chloride solution (5 ml). The phases are separated and the organic phase is dehydrated with magnesium sulphate and concentrated under vacuum to a residue. About 52 g of product are obtained as thick oil.

### Example 7 (Comparative)

Waste Fulvestrant (5 g, 8.24 mmol) is loaded into a 3-necked flask equipped with a mechanical stirrer, thermometer and condenser. THF (25 ml) is charged under nitrogen and there is complete dissolution. Sodium sulfite (1.14 g, 8.8 mmol) and iodine (0.21 g, 0.82 mmol) are added to the solution. The reaction mixture is heated under reflux and monitored over time. The reaction is slow, after 23 h the conversion is 66%. The reaction is unsatisfactory and is not processed.

### Examples of Oxidation from Sulfide to Sulfoxide

### Example 8

Sulfide (II) (20 g, 33.8 mmol obtained from example 3) is charged into a 500 ml 3-necked flask, equipped with a mechanical stirrer, thermometer, and under nitrogen atmosphere, and dissolved in THF (235 ml) and methanol (60 ml). It is cooled to 5° C. and a solution of sodium (meta) periodate (11.3 g, 49 mmol) in water (80 ml) is added. It is left to react at room temperature for 15-18 h. At the end of the reaction, the organic solvents are removed by distilling under vacuum and methylene chloride (200 ml) is added. The phases are separated. It is washed with water, the solvent is removed under vacuum and crystallized from toluene to obtain crude Fulvestrant (13 g).

Crude Fulvestrant is then purified by silica chromatography (300 g, eluent: methylene chloride/methanol 98:2) and the cleaned fractions are crystallized from ethyl acetate. 9.4 g of product are obtained in compliance with the specifications according to the pharmacopoeias. The order of elution is: sulfide, sulfone containing waste Fulvestrant, product Fulvestrant.

1. A process for converting waste Fulvestrant having formula (I) 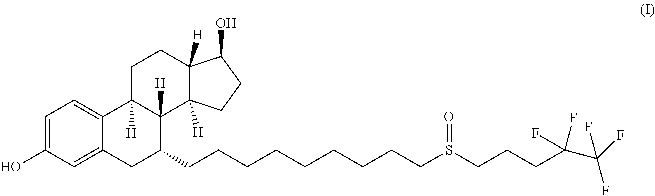
to a sulfide having formula (II) 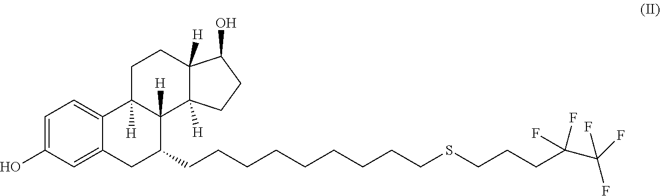
the process comprising reducing the waste Fulvestrant (I) to the sulfide having formula (II) in presence of a reducing agent selected from the group consisting of sodium bisulfite, sodium metabisulfite, and diiron-nonacarbonyl phenylsilane.

2. The process according to claim 1, wherein when said reducing agent is sodium bisulfite or sodium metabisulfite, a catalytic amount of iodine is added.

3. The process according to claim 1, wherein said reducing agent is used as a solid or as an aqueous solution.

4. The process according to claim 3, wherein said reducing agent is used as an aqueous solution.

5. The process according to claim 4, wherein said aqueous solution has a concentration of the reducing agent of between 10% and 45%

6. The process according to claim 4, wherein said aqueous solution has a concentration of the reducing agent of about 40%.

7. The process according to claim 1, wherein said reducing agent is used in a molar amount of between 1.0 and 2.0 equivalents with respect to the molar amount of the waste Fulvestrant.

8. The process according to claim 7, wherein said reducing agent is used in a molar amount of between 1.0 and 1.5 equivalents with respect to the molar amount of the waste Fulvestrant.

9. The process according to claim 1, further comprising an oxidation reaction to convert the sulfide (II) formed during the recycling step into additional Fulvestrant.

10. The process according to claim 9, wherein said oxidation reaction is performed in the presence of an oxidizing reagent selected from the group consisting of sodium (meta)periodate and meta chloroperbenzoic acid.

12. In a process comprising oxidizing a sulfide having formula (II) 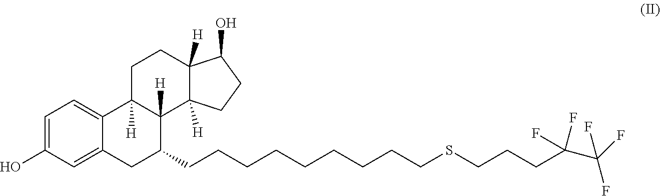
to produce product Fulvestrant having formula (I) 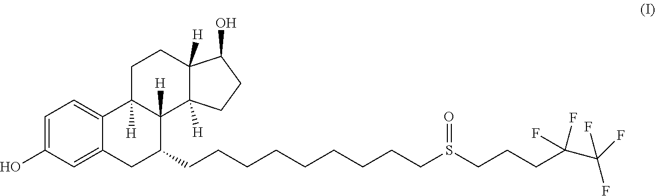
wherein product Fulvestrant exists as a mixture of two epimeric diastereomers at the sulfur atom known as diastereomer A and diastereomer B in a ratio of 42-48/58-52;
wherein the process also produces waste Fulvestrant having a ratio of diastereomer A and diastereomer B outside of 42-48/58-52; and
wherein the waste Fulvestrant is recycled by a reduction reaction to obtain the corresponding sulfide having formula (II) and subsequent oxidation to form additional product Fulvestrant;
the improvement wherein the reduction of the waste Fulvestrant during the recycling step takes place in the presence of a reducing agent selected from one or more of sodium bisulfate, sodium metabisulfite, and diiron-nonacarbonyl.
